(19) United States
(12) Patent Application Publication
(10) Pub. No.: US 2023/0233808 A1
(43) Pub. Date: Jul. 27, 2023

(54) CATHETER ASSEMBLY AND RELATED METHODS

(71) Applicant: Becton, Dickinson and Company, Franklin Lakes, NJ (US)

(72) Inventor: Kevin Ryan, Whitehouse Station, NJ (US)

(21) Appl. No.: 18/075,188

(22) Filed: Dec. 5, 2022

Related U.S. Application Data
(60) Provisional application No. 63/302,425, filed on Jan. 24, 2022.

(51) Int. Cl. A61M 25/06; A61M 25/00

(52) U.S. Cl. CPC A61M 25/0606; A61M 25/0012; A61M 25/0097

(57) ABSTRACT

A catheter assembly may include a catheter hub, which may include a distal end, a proximal end, and a catheter hub lumen extending through the distal end of the catheter hub and the proximal end of the catheter hub. The catheter assembly may include a catheter extending from the distal end of the catheter hub. The catheter may include a catheter tube, which may include a distal end, a proximal end, and a catheter tube lumen extending through the distal end of the catheter tube and the proximal end of the catheter tube. The catheter may also include a fluid-permeable section extending from the distal end of the catheter tube. The fluid-permeable section may include a coil or a lattice. The catheter assembly may include an introducer needle extending through the catheter tube and the fluid-permeable section.

### RELATED APPLICATIONS

This application claims the benefit of U.S. Provisional Patent Application No. 63/302,425, filed Jan. 24, 2022, and entitled CATHETER ASSEMBLY AND RELATED METHODS, which is incorporated herein in its entirety.

### BACKGROUND

A common type of catheter assembly includes a peripheral intravenous catheter (“PIVC”) that is over-the-needle. As its name implies, the PIVC that is over-the-needle may be mounted over an introducer needle having a sharp distal tip. The catheter assembly may include a catheter hub, the PIVC extending distally from the catheter hub, and the introducer needle extending through the PIVC. The PIVC and the introducer needle may be assembled such that the distal tip of the introducer needle extends beyond the distal tip of the PIVC with the bevel of the needle facing up away from skin of the patient immediately prior to insertion into the skin. The PIVC and the introducer needle are generally inserted at a shallow angle through the skin into vasculature of the patient.

In order to verify proper placement of the introducer needle and/or the PIVC in the blood vessel, a clinician may confirm that there is flashback of blood in a flashback chamber of the catheter assembly. Once placement of the introducer needle has been confirmed, the clinician may remove the introducer needle, leaving the PIVC in place for future blood withdrawal or fluid infusion.

PIVC's often only remain open to flow for a few days and may require removal due to failure prior to their clinical need being fulfilled. In further detail, when the PIVC is inserted into a vein of a patient, it often obstructs blood flow through the vein and creates stasis, which overtime can lead to platelet and fibrin deposition and thrombosis at the distal tip of the PIVC. Also, overtime infusion into the vein via the PIVC contributes to excessive Wall Shear Stress (“WSS”), which causes venous wall damage, leading to phlebitis and thrombosis. As a result of the PIVC's obstruction of flow through the vein and/or infusion, the PIVC may need to be removed prior to completion of infusion and/or blood draw.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices and related methods. More particularly, the present disclosure relates to a catheter assembly and related methods. In some embodiments, a catheter assembly may include a catheter hub, which may include a distal end, a proximal end, and a catheter hub lumen extending through the distal end of the catheter hub and the proximal end of the catheter hub. In some embodiments, the catheter assembly may include a catheter extending from the distal end of the catheter hub. In some embodiments, the catheter may include a catheter tube, which may include a distal end, a proximal end, and a catheter tube lumen extending through the distal end of the catheter tube and the proximal end of the catheter tube.

In some embodiments, the catheter may also include a fluid-permeable section extending from the distal end of the catheter tube. In some embodiments, the fluid-permeable section may include a coil or a mesh. In some embodiments, the catheter assembly may include an introducer needle extending through the catheter tube and the fluid permeable section.

In some embodiments, the catheter tube may be over-molded with the coil or the mesh. In some embodiments, the fluid-permeable section may be constructed of a different material than the catheter tube. In some embodiments, the fluid-permeable section may be constructed of a polymer material or metal. In some embodiments, the coil or the mesh may extend along an entire length of the catheter tube. In some embodiments, the coil or the mesh may extend along a partial length of the catheter tube such that a proximal end of the coil or the mesh is spaced apart from a proximal end of the catheter tube.

In some embodiments, the fluid-permeable section may be formed of a same material as the catheter tube. In some embodiments, the distal end of the catheter tube may include a bevel. In some embodiments, a distal end of the introducer needle may include another bevel. In some embodiments, the bevel of the catheter tube may face in an opposite direction as the other bevel of the introducer needle.

In some embodiments, the fluid-permeable section may include one or more clip features and an outer surface of the introducer needle may include one or more corresponding clip features. In some embodiments, the one or more clip features and the one or more corresponding clip features contact each other. In some embodiments, the one or more clip features and the one or more corresponding clip features may be configured to prevent a distal end of the fluid-permeable section from moving proximal to the one or more corresponding clip features. In some embodiments, each of the one or more clip features may include a groove. In some embodiments, each of the one or more clip features may include a protrusion. In some embodiments, the distal end of the fluid-permeable section may include an annular ring. In some embodiments, the annular ring may include the one or more clip features.

In some embodiments, the catheter tube may include multiple slits forming multiple semi-annular sections. In some embodiments, in response to bending of the catheter tube, at least some of the multiple semi-annular sections may be configured to separate to create a fluid pathway from an exterior of the catheter tube to the catheter tube lumen. In some embodiments, the multiple semi-annular sections may be configured to contact each other when the catheter is straight or not subjected to any external load forces. In some embodiments, the introducer needle may extend through the catheter tube and may include a bevel. In some embodiments, each of the multiple slits may extend partially through the catheter tube to maintain a continuous surface aligned with the bevel of the introducer needle. In some embodiments, the multiple semi-annular sections may be a same size.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is an upper perspective view of an example catheter assembly, according to some embodiments;

FIG. 2A is an upper perspective view of an example needle assembly, according to some embodiments;

FIG. 2B is an upper perspective view of an example catheter hub and an example catheter tube, according to some embodiments;

FIG. 2C is a cross-sectional view of the catheter hub and the catheter tube, according to some embodiments;

FIG. 3A is an upper perspective view of an example catheter, according to some embodiments;

FIG. 3B is an upper perspective view of an example mesh, according to some embodiments;

FIG. 3C is an upper perspective view of the catheter, illustrating the mesh and the catheter tube constructed of a same material;

FIG. 3D is an upper perspective view of the catheter, illustrating an example closed coil, according to some embodiments;

FIG. 3E is an upper perspective view of the catheter, illustrating an example open coil, according to some embodiments;

FIG. 4A is an upper perspective view of an example distal end of the catheter, according to some embodiments;

FIG. 4B is a cross-sectional view of the distal end of the catheter, according to some embodiments;

FIG. 5A is a side view of the catheter assembly inserted into vasculature of a patient and in a fluid delivery or blood withdrawal configuration, according to some embodiments;

FIG. 5B is a side view of the catheter assembly being inserted into the patient with an example introducer needle, according to some embodiments;

FIG. 5C is a side view of the catheter assembly after some distal advancement of the catheter hub with respect to the introducer needle, according to some embodiments;

FIG. 5D is an enlarged view of a portion of the catheter assembly of FIG. 5A, according to some embodiments;

FIG. 6A is a side view of another example catheter, according to some embodiments; and

FIG. 6B is another side view of the other example catheter, according to some embodiments; and

FIG. 6C is a cross-sectional view of the other example catheter along the line 6C-6C of FIG. 6A, according to some embodiments.

### DESCRIPTION OF EMBODIMENTS

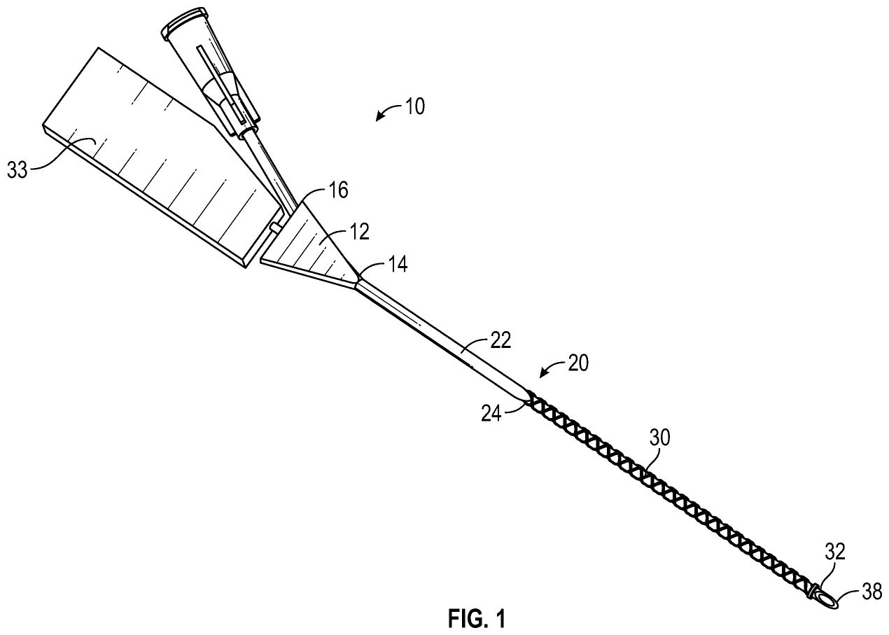

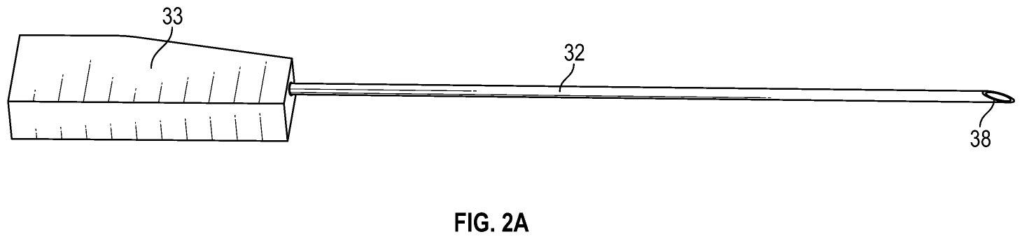

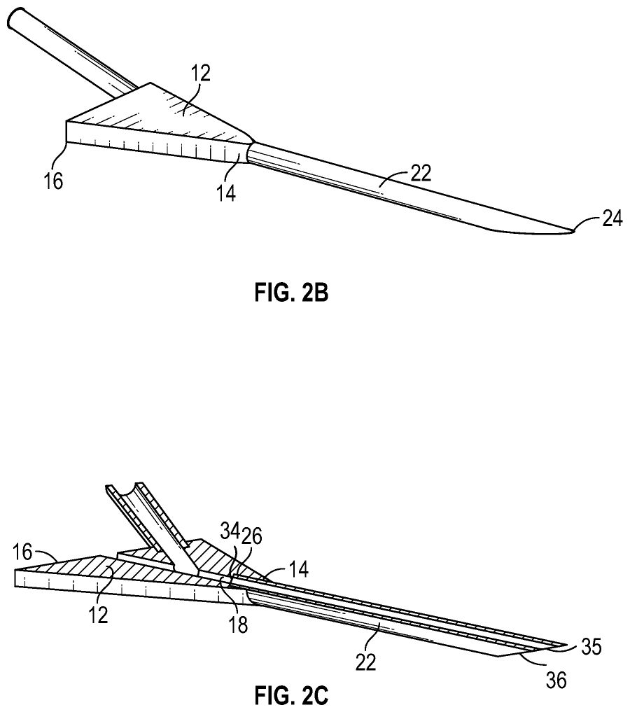

Referring now to FIG. 1-2C, in some embodiments, a catheter assembly 10 may include a catheter hub 12, which may include a distal end 14, a proximal end 16, and a catheter hub lumen 18 extending through the distal end 14 of the catheter hub 12 and the proximal end 16 of the catheter hub 12. In some embodiments, the catheter assembly 10 may include a catheter 20 extending from the distal end 14 of the catheter hub 12. In some embodiments, the catheter 20 may include a peripheral intravenous catheter (PIVC), a midline catheter, or a peripherally-inserted central catheter. In some embodiments, the catheter 20 may include a catheter tube 22, which may include a distal end 24, a proximal end 26, and a catheter tube lumen 28 extending through the distal end 24 of the catheter tube 22 and the proximal end 26 of the catheter tube 22.

In some embodiments, the catheter 20 may also include a fluid-permeable section 30 extending from the distal end 24 of the catheter tube 22. A traditional catheter tube without the fluid-permeable section 30 causes blood to flow around the traditional catheter tube within a vein of a patient, restricting blood flow within the vein and increasing a likelihood of platelet and fibrin deposition and thrombosis at a tip of the traditional catheter. In some embodiments, in contrast to the traditional catheter tube, when the catheter assembly 10 is inserted into the vein of the patient for fluid delivery or blood withdrawal, the distal end 24 of the catheter tube 22 may be positioned just inside or just outside the vein, and the fluid-permeable section 30 within the vein may facilitate fluid flow within the vein. Thus, in some embodiments, the fluid-permeable section 30 may lower blood flow restriction compared to the traditional catheter, reducing platelet and fibrin deposition on the catheter 20. The fluid-permeable section 30 may thereby lengthen a dwell time of the catheter 20, increasing a period of time that the catheter 20 is functional.

In some embodiments, the fluid-permeable section 30 further allows a clinician to use a larger catheter tube without negatively impacting blood flow restriction through the vein. Moreover, the fluid-permeable section 30 may maintain structural integrity of the vein, providing a stent-like function and holding the vein open and prolonging an open flow path for blood flow. In some embodiments, the fluid-permeable section 30 may thus facilitate blood flow from the vein through the fluid-permeable section 30 and into the catheter tube 22 and the catheter assembly 10.

In some embodiments, the fluid-permeable section 30 may include a mesh. As used in the present disclosure, the term “mesh” is intended to include any element having an openwork fabric or structure, and may include, but is not limited to, an interconnected network of wire-like segments, a sheet of material having numerous apertures and/or portions removed, or the like. In some embodiments, the mesh may be constructed of metal, a polymer material, or another suitable material such that the mesh is configured to bend or flex when inserted into the vein. In some embodiments, the metal may include stainless steel or another suitable metal. In some embodiments, the polymer may include polyurethane or another suitable polymer. In some embodiments, the mesh may include a lattice, where a geometric space, such as a square or a diamond, is repeated in the circumferential and axial directions of the mesh. In some embodiments, the mesh may be cylindrical and configured to fit within the vein of the patient, with a multiplicity of openings to facilitate fluid flow therethrough. In some embodiments, the fluid-permeable section 30 may include a coil or another suitable shape configured to facilitate fluid flow therethrough at least when the fluid-permeable section 30 is in a bent position.

As illustrated in FIG. 2A, an introducer needle 32 may extend from a needle hub 33. In some embodiments, the needle hub 33 may include a flashback chamber and/or a needle safety device. As illustrated in FIG. 1, in some embodiments, the introducer needle 32 may extend through the catheter hub 12 and the catheter 20 when the catheter assembly 10 is in an insertion configuration ready for insertion into the patient. In some embodiments, the introducer needle 32 may include a sharp distal tip configured to pierce skin and the vein of the patient. In some embodiments, after the introducer needle 32 is used to place the catheter tube 22 and/or the fluid-permeable section 30 within the vein, the catheter 20 may be advanced distally further into the vein, and the introducer needle 32 may then be removed from the catheter 20 and the catheter hub 12. In some embodiments, the catheter 20 may be advanced distally until the catheter hub 12 contacts or buts up against the skin of the patient.

In some embodiments, the distal end 24 of the catheter tube 22 may include a bevel 36. In some embodiments, the sharp distal tip of the introducer needle 32 may include another bevel 38. In some embodiments, the bevel 36 of the catheter tube 22 may face in an opposite direction as the other bevel 38 of the introducer needle 32. Thus, in some embodiments, the bevel 36 of the catheter tube 22 may be oriented generally parallel to the vein, which may facilitate blood flow through the catheter tube 22.

In some embodiments, the catheter tube 22 may be shorter than the traditional catheter tube and may thus cause less blood flow restriction in the vein. In further detail, in some embodiments, after the introducer needle 32 has been removed and the catheter 20 is placed in a fluid delivery or blood withdrawal configuration with the catheter hub 12 contacting or butting up against the skin of the patient, the catheter tube 22 may be disposed just outside of the vein or just inside the vein. In some embodiments, because the catheter tube 22 is disposed just outside or just inside the vein, blood flow may be less restricted within the vein compared to the traditional catheter and the catheter 20 may be used for a longer period of time without failure. In some embodiments, the catheter tube 22 may be long enough such that it forms a seal with an incision hole extending through the skin of the patient when the catheter 20 is in the fluid delivery or blood withdrawal configuration with the catheter hub 12 contacting or butting up against the skin of the patient. In these and other embodiments, the catheter tube 22 may extend through the skin at an insertion angle between 10° and 30° when in the fluid delivery or blood withdrawal configuration, ready for infusion or blood collection.

As illustrated in FIGS. 2B-2C, in some embodiments, the catheter tube 22 may be closed except for a distal opening 35 in the distal end 24 and a proximal opening 34 in the proximal end 26, which may each be aligned with a longitudinal axis of the catheter tube 22. In further detail, the catheter tube 22 may include an annular closed wall, the distal opening 35, and the proximal opening 34. Thus, there may be little or no leakage of fluid from the catheter tube 22 proximal to the distal opening 35. FIGS. 2B-2C illustrate the catheter 20 without the mesh or the coil for illustrative purposes.

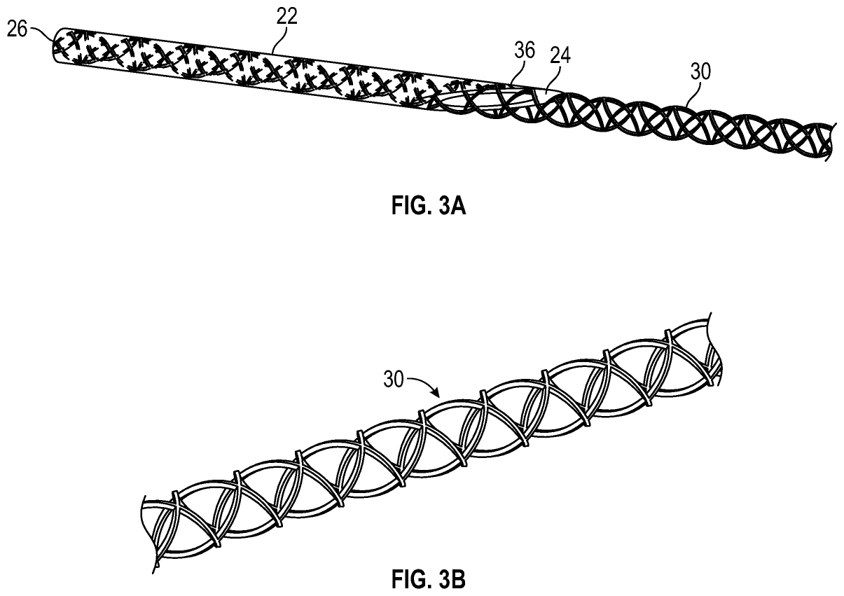

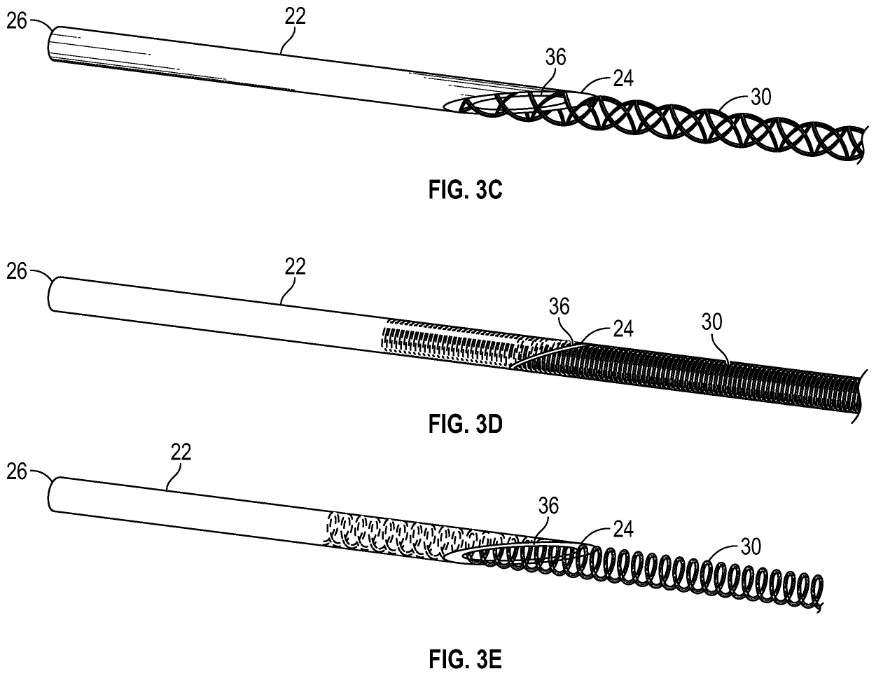

Referring now to FIGS. 3A-3B, in some embodiments, the fluid-permeable section 30 may be constructed of a different material than the catheter tube 22. In some embodiments, the fluid-permeable section 30 may be constructed of one or more of a metal, a polymer, or another suitable material configured to bend or flex when inserted into the vein. In some embodiments, the metal may include stainless steel or another suitable metal. In some embodiments, the polymer may include polyurethane or another suitable polymer. In some embodiments, the fluid-permeable section 30 may be constructed of a more flexible material than the catheter tube 22 since the fluid-permeable section 30 may bend within the vein when the catheter assembly 10 is in the fluid delivery or blood withdrawal configuration. In some embodiments, the catheter tube 22 may be constructed of one or more of polyethylene, silastic, plastic, a polymer, or another suitable material.

In some embodiments, the catheter tube 22 may be over-molded with the mesh or the coil. In some embodiments, over-molding of the catheter tube 22 and the mesh or the coil may increase flexibility at an exit of the mesh from the catheter tube 22, provide resistance to impact, and provide strain relief. As illustrated in FIG. 3A, in some embodiments, the mesh may extend along an entire length of the catheter tube 22, which may enhance benefits of the over-molding. In other embodiments, the mesh may extend along a partial length of the catheter tube 22 such that a proximal end of the mesh is spaced apart from a proximal end 26 of the catheter tube 22.

Referring now to FIG. 3C, in some embodiments, the fluid-permeable section 30, such as the mesh or the coil, may be formed of a same material as the catheter tube 22. In these embodiments, the fluid-permeable section 30 and the catheter tube 22 may be monolithically formed as a single unit. In further detail, a tube longer than the catheter tube 22 may be monolithically formed as a single unit, and cuts may be made in the tube to remove portions of the tube and form the fluid-permeable section 30. In some embodiments, the cuts may be made with a laser or another suitable device. In some embodiments, the fluid-permeable section 30 and the catheter tube 22 may be monolithically formed as a single unit via injection molding.

Referring now to FIG. 3D, in some embodiments, the fluid-permeable section 30 may include the coil, which may be closed. In further detail, in some embodiments, there may not be significant gaps between individual loops of the coil or the individual loops may contact each other when the fluid-permeable section 30 is straight or not subjected to any external load forces. In these embodiments, the coil may be similar to a fully compressed tension spring. However, in some embodiments, when the coil is bent or curved, such as when a proximal portion of the coil is disposed at the insertion angle and a distal portion of the coil is disposed at a vein direction angle within the vein, some of the coils may open or expand. In some embodiments, opening or expanding of the coils may increase blood flow from the vein into the coil and into the catheter assembly 10.

Referring now to FIG. 3E, the fluid-permeable section 30 may include the coil, which may be open. In further detail, in some embodiments, there may gaps between the individual loops or the individual loops may not contact each other when the fluid-permeable section 30 is straight or not subjected to any external load forces. In some embodiments, the coil that is open may lower blood flow restriction within the vein compared to the traditional catheter, prolonging a life of the catheter 20.

Referring now to FIGS. 3D-3E, in some embodiments, the coil may be over-molded with the catheter tube 22. In some embodiments, the coil may extend along an entire length of the catheter tube 22, which may increase flexibility at an exit of the coil from the catheter tube 22, provide resistance to impact, and provide strain relief In other embodiments, the coil may extend along a partial length of the catheter tube 22 such that a proximal end of the coil is spaced apart from a proximal end 26 of the catheter tube 22.

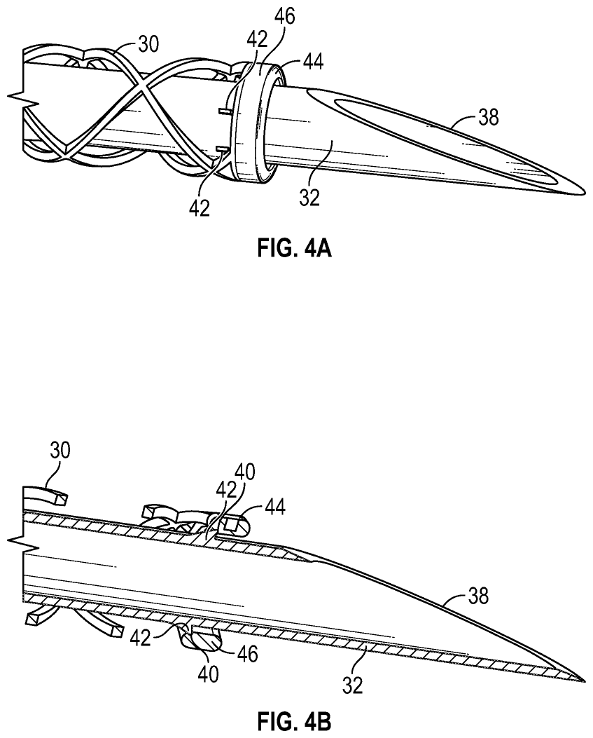

Referring now to FIGS. 4A-4B, in some embodiments, the fluid-permeable section 30, such as the mesh or the coil, may include one or more clip features 40 and an outer surface of the introducer needle 32 may include one or more corresponding clip features 42. In some embodiments, the one or more clip features 40 and the one or more corresponding clip features 42 may be coupled together, contacting each other. In some embodiments, the one or more clip features 40 and the one or more corresponding clip features 42 may be configured to prevent a distal end 44 of the fluid-permeable section 30 from moving proximal to the one or more corresponding clip features 42. For example, when the introducer needle 32 and the fluid-permeable section 30 are pushed through the skin and/or the vein, coupling between the one or more clip features 40 and the one or more corresponding clip features 42 may prevent the fluid-permeable section 30 from getting caught or bunched.

In further detail, in some embodiments, each of the one or more clip features 40 may include a groove, as illustrated, for example, in FIG. 4B. In some embodiments, each of the one or more corresponding clip features 42 may include a protrusion, as illustrated, for example, in FIG. 4B. In some embodiments, the protrusion may include a distal stop surface that contacts the one or more clip features 40 and to which force is applied by the one or more clip features 40 during insertion of the catheter 20 and the introducer needle 32 into the patient.

In some embodiments, the one or more clip features 40 and the one or more corresponding clip features 42 may be configured to allow easy proximal retraction of the introducer needle 32 within respect to the fluid-permeable section 30 and the catheter 20. In some embodiments, an outer diameter of the introducer needle 32 at the one or more corresponding clip features 42 may be less than an inner diameter of the fluid-permeable section 30 to facilitate easy proximal retraction of the introducer needle 32 after the catheter 20 is inserted within the vein.

In some embodiments, the distal end 44 of the fluid-permeable section 30 may include an annular ring 46, which may facilitate easy insertion of the fluid-permeable section 30 into the patient. In some embodiments, the annular ring 46 may include the one or more clip features 40.

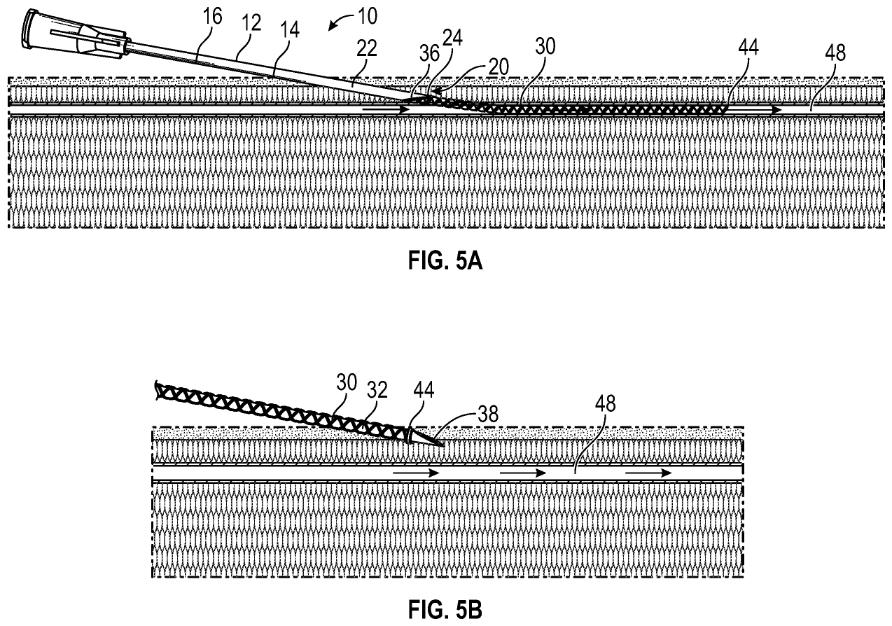

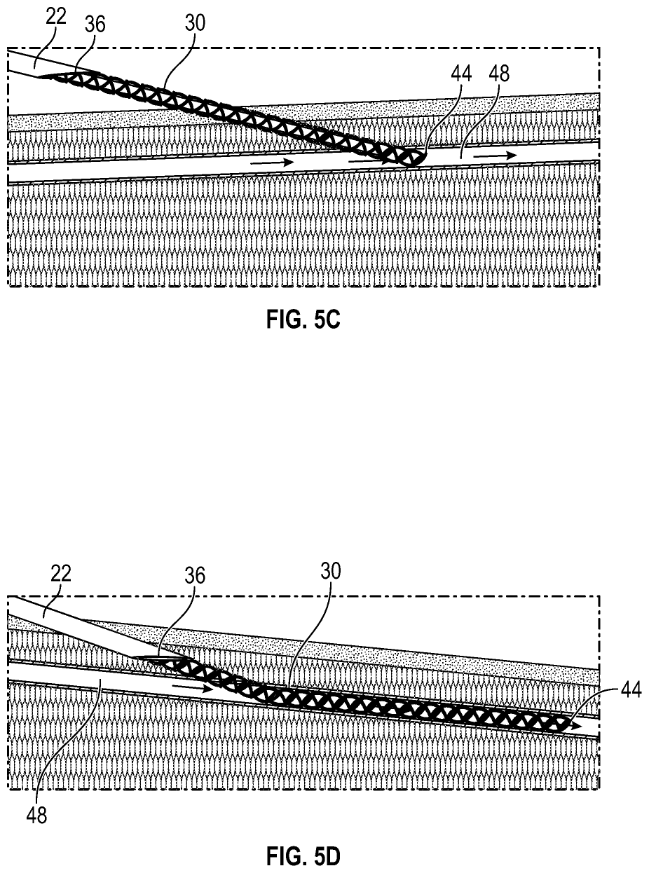

Referring now to FIG. 5A, a side view of the catheter assembly 10 inserted into vasculature of the patient and in a fluid delivery or blood withdrawal configuration is illustrated, according to some embodiments. Referring now to FIG. 5B, the catheter assembly 10 is illustrated being inserted into the patient with the introducer needle 32, according to some embodiments. Referring now to FIG. 5C, the catheter assembly 10 is illustrated after some distal advancement of the catheter hub 12 with respect to the introducer needle 32, according to some embodiments. FIG. 5D is an enlarged view of a portion of the catheter assembly 10 of FIG. 5A, according to some embodiments.

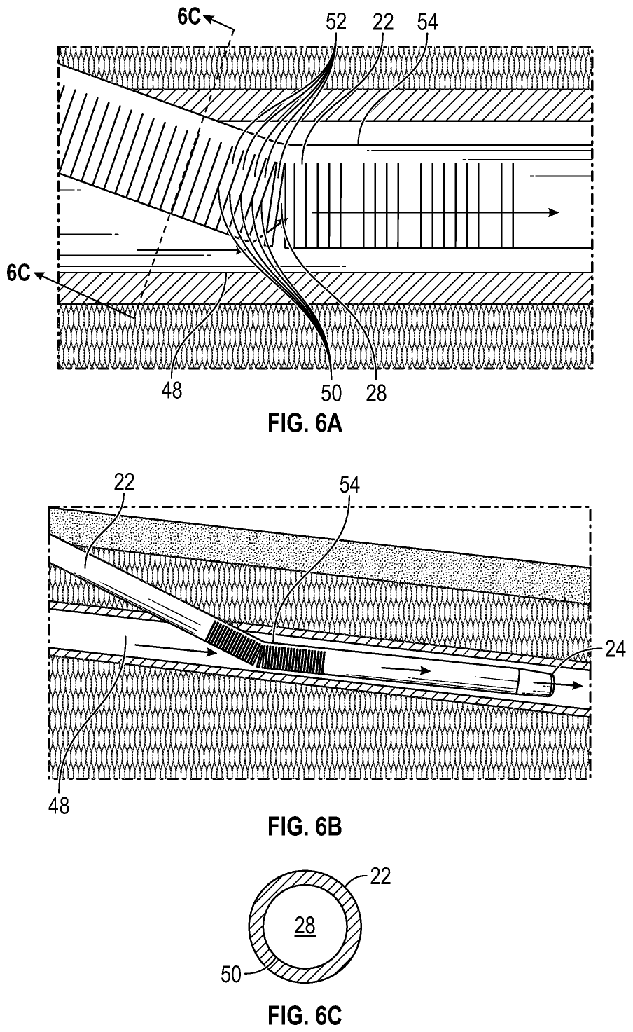

Referring now to FIGS. 6A-6B, in some embodiments, the catheter tube 22 may include multiple slits 50 forming multiple semi-annular sections 52. In some embodiments, in response to bending of the catheter tube 22, at least some of the multiple semi-annular sections 52 may be configured to separate to create a fluid pathway from an exterior of the catheter tube 22 to the catheter tube lumen 28. In some embodiments, the multiple semi-annular sections 52 may be configured to contact each other when the catheter tube 22 is straight or not subjected to any external load forces. In some embodiments, a particular semi-annular section 52 in between two other particular semi-annular sections 52 may contact an entirety of the two other particular semi-annular sections 52 when the catheter tube 22 is straight or not subjected to any external load forces, such that little to no fluid passes between the particular semi-annular section 52 and the two other particular semi-annular sections 52. In some embodiments, the introducer needle 32 may extend through the catheter tube 22 and may include the other bevel 38 (see, for example, FIG. 1). In some embodiments, each of the multiple slits may extend partially through the catheter tube 22 to maintain a continuous surface 54 aligned with the other bevel 38 of the introducer needle 32. In some embodiments, the multiple semi-annular sections 52 may be a same size as each other, which may facilitate low or no fluid flow into the catheter tube 22 when the catheter tube 22 is straight or not subjected to any external load forces.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter assembly, comprising:
a catheter hub, comprising a distal end, a proximal end, and a catheter hub lumen extending through the distal end of the catheter hub and the proximal end of the catheter hub;
a catheter extending from the distal end of the catheter hub, wherein the catheter comprises:
a catheter tube comprising a distal end, a proximal end, a catheter tube lumen extending through the distal end of the catheter tube and the proximal end of the catheter tube; and
a fluid-permeable section extending from the distal end of the catheter tube, wherein the fluid-permeable section comprises a mesh or a coil; and
an introducer needle extending through the catheter tube and the fluid-permeable section.

2. The catheter assembly of claim 1, wherein the catheter tube is over-molded with the mesh or the coil.

3. The catheter assembly of claim 2, wherein the fluid-permeable section is constructed of a polymer material.

4. The catheter assembly of claim 2, wherein the fluid-permeable section is constructed of metal.

5. The catheter assembly of claim 2, wherein the mesh or the coil extends along an entire length of the catheter tube.

6. The catheter assembly of claim 2, wherein the mesh or the coil extends along a partial length of the catheter tube such that a proximal end of the mesh or the coil is spaced apart from a proximal end of the catheter tube.

7. The catheter assembly of claim 1, wherein the fluid-permeable section is formed of a same material as the catheter tube.

8. The catheter assembly of claim 1, wherein the distal end of the catheter tube comprises a bevel.

9. The catheter assembly of claim 8, wherein a distal end of the introducer needle comprises another bevel, wherein the bevel of the catheter tube faces in an opposite direction as the other bevel of the introducer needle.

10. The catheter assembly of claim 1, wherein the fluid-permeable section is constructed of a different material than the catheter tube.

11. The catheter assembly of claim 1, wherein the fluid-permeable section comprises a plurality of clip features and an outer surface of the introducer needle comprises a plurality of corresponding clip features, wherein the plurality of clip features and the plurality of corresponding clip features contact each other and are configured to prevent a distal end of the fluid-permeable section from moving proximal to the plurality of corresponding clip features.

12. The catheter assembly of claim 11, wherein each of the plurality of clip features comprises a groove, wherein each of the plurality of clip features comprises a protrusion.

13. The catheter assembly of claim 11, wherein the distal end of the fluid-permeable section comprises an annular ring, wherein the annular ring comprises the plurality of clip features.

14. The catheter assembly of claim 1, wherein the distal end of the fluid-permeable section comprises an annular ring.

15. The catheter assembly of claim 1, wherein the fluid-permeable section comprises the coil.

16. The catheter assembly of claim 1, wherein the fluid-permeable section comprises the mesh.

17. A catheter assembly, comprising:
a catheter hub, comprising a distal end, a proximal end, and a catheter hub lumen extending through the distal end of the catheter hub and the proximal end of the catheter hub; and
a catheter tube extending from the distal end of the catheter hub, wherein the catheter tube comprises a distal end, a proximal end, a catheter tube lumen extending through the distal end of the catheter tube and the proximal end of the catheter tube, and a plurality of slits forming a plurality of semi-annular sections, wherein in response to bending of the catheter tube, at least some of the plurality of semi-annular sections are configured to separate to create a fluid pathway from an exterior of the catheter tube to the catheter tube lumen.

18. The catheter assembly of claim 17, wherein the plurality of semi-annular sections are configured to contact each other when the catheter tube is straight.

19. The catheter assembly of claim 17, further comprising an introducer needle extending through the catheter tube and comprising a bevel, wherein each of the plurality of slits extends partially through the catheter tube to maintain a continuous surface aligned with a bevel of the introducer needle.

20. The catheter assembly of claim 17, wherein the plurality of semi-annular sections are a same size.
